(19) United States
(12) Patent Application Publication
(10) Pub. No.: US 2004/0034417 A1
(43) Pub. Date: Feb. 19, 2004

(54) Intraocular lens

(76) Inventor: Thomas M. Heyman, Mansfield, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/222,149

(22) Filed: Aug. 16, 2002

(51) Int. Cl. A61F 2/16

(52) U.S. Cl. 623/6.46; 623/6.49

(57) ABSTRACT

A foldable lens having a plurality of generally “T”-shaped haptics that vault the lens posteriorly when implanted in the eye while still providing stable fixation of the lens within the eye. Such a construction ensures firm contact between the optic and the posterior capsule while still allowing the anterior capsule and the posterior capsule to contact and cause fibrosis about the haptics.

This invention relates to intraocular lenses (IOLs) and more particularly to soft, foldable intraocular lenses.

### BACKGROUND OF THE INVENTION

The human eye in its simplest terms functions to provide vision by transmitting and refracting light through a clear outer portion called the cornea, and further focusing the image by way of lens onto the retina at the back of the eye. The quality of the focused image depends on many factors including the size, shape and length of the eye, and the shape and transparency of the cornea and lens.

When trauma, age or disease cause the lens to become less transparent, vision deteriorates because of the diminished light which can be transmitted to the retina. This deficiency in the lens of the eye is medically known as a cataract. The treatment for this condition is surgical removal of the lens and implantation of an artificial lens or IOL.

While early IOLs were made from hard plastic, such as polymethylmethacrylate (PMMA), soft foldable IOLs made from silicone, soft acrylics and hydrogels have become increasingly popular because of the ability to fold or roll these soft lenses and insert them through a smaller incision. While early foldable lenses either had a plate-style haptic (e.g., U.S. Pat. No. 4,664,666 (Barrett), the entire contents of which being incorporated herein by reference) or were of a multi-piece design with independently formed, relatively rigid haptic attached to the soft optic (e.g., U.S. Pat. No. 5,118,452 (Lindsey, et al.), the entire contents of which being incorporated herein by reference), newer lens designs are of an open-loop variety and manufactured from a single piece (e.g., U.S. Pat. No. 5,716,403, (Tran, et al.), the entire contents of which being incorporated herein by reference). The problem with current soft, planar haptic, single-piece, open loop IOLs is that the haptics lack axial force to vault the optic posteriorly away from the iris and ensure firm contact with the posterior capsule. With respect to single piece, closed loop, vaulted soft IOLs, one reference, U.S. Pat. No. 6,409,762 B1 (Pynson, et al.), the entire contents of which being incorporated herein by reference, suggests that such a construction is undesirable due to tilt and unpredictable axial displacement of the lens in the eye.

Accordingly, a need continues to exist for a vaulted, single-piece, open loop, soft intraocular lens.

### BRIEF SUMMARY OF THE INVENTION

The present invention improves upon prior art single-piece, open loop, soft intraocular lenses by providing a foldable lens having a plurality of generally “T”-shaped haptics that vault the lens posteriorly when implanted in the eye while still providing stable fixation of the lens within the eye. Such a construction ensures firm contact between the optic and the posterior capsule while still allowing the anterior capsule and the posterior capsule to contact and cause fibrosis about the haptics.

It is accordingly an object of the present invention to provide a stable intraocular lens.

It is a further object of the present invention to provide a vaulted, single-piece, open loop, soft intraocular lens.

It is a further object of the present invention to provide a single-piece, open loop, soft intraocular lens having a plurality of generally “T”-shaped haptics that vault the lens posteriorly when implanted in the eye.

Other objectives, features and advantages of the present invention will become apparent with reference to the drawings, and the following description of the drawings and claims.

### BRIEF DESCRIPTION OF THE DRAWINGS

FIG. 1 is a top plan view of a first embodiment of the intraocular lens of the present invention.

FIG. 2 is a side elevational view of a first embodiment of the intraocular lens of the present invention.

FIG. 3 is a perspective view a first embodiment of the intraocular lens of the present invention.

FIG. 4 is a perspective view a second embodiment of the intraocular lens of the present invention.

FIG. 5 is a perspective view a third embodiment of the intraocular lens of the present invention.

### DETAILED DESCRIPTION OF THE INVENTION

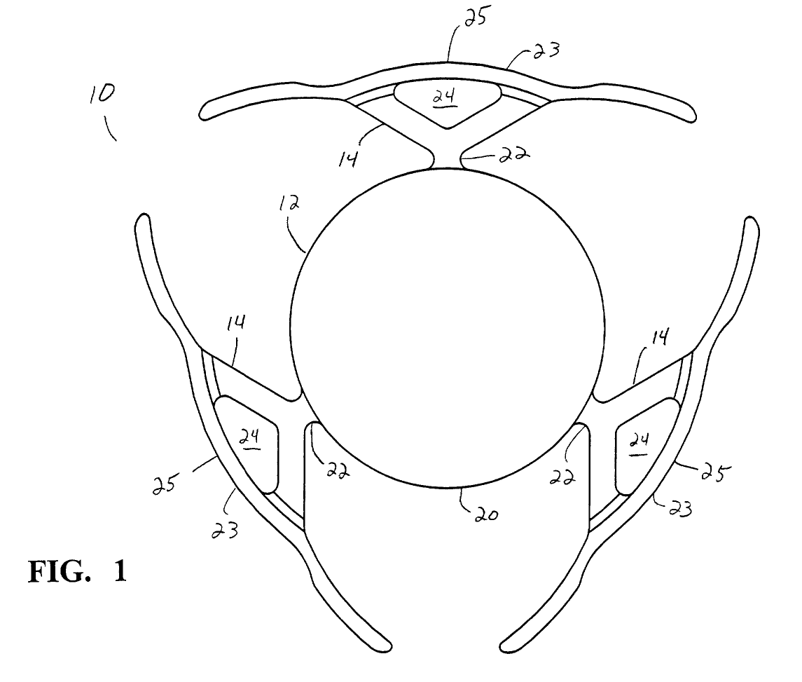

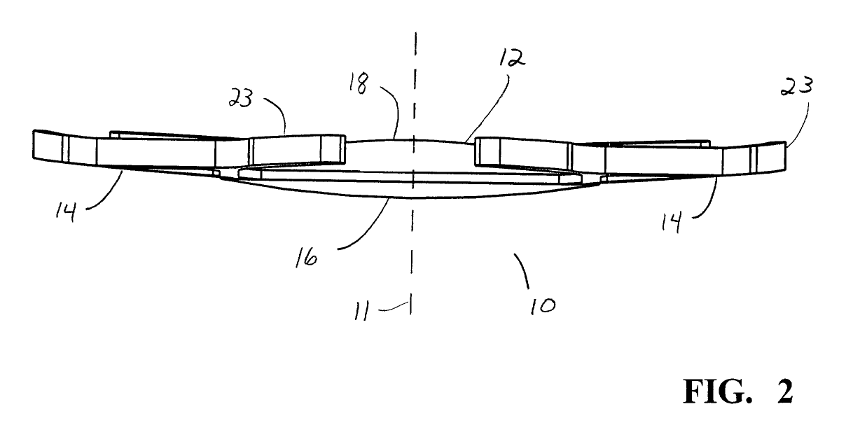

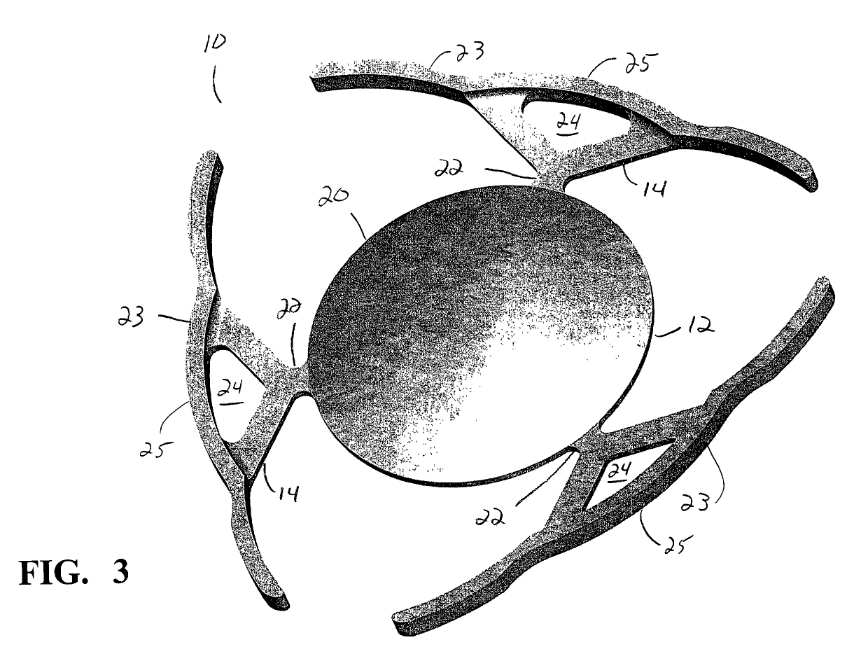

As best seen in FIGS. 1 and 2, lens 10 generally consists of optic 12 and a plurality of haptics 14. Optic 12 has an anterior face 18 and a posterior face 16. Lens 10 may have any suitably designed optic 12 (e.g., multifocal, toric, monofocal) and preferably made in a single piece from a soft, foldable material, such as silicone, hydrogel or soft acrylic and symmetrical about optical axis 11. Haptics 14 intersects edge 20 of optic 12 at an angle relative to the plane of the optic, preferably between 1° and 20°, and most preferably between 3° and 10° but other suitable angles may also be used. Haptics 14 preferably are generally “T”-shaped in the plane of optic 12, attached to optic 12 at joints 22 and contain open areas 24 that reduce the mass and increase the flexibility of haptics 14. Open areas 24 also provide for increased contact between the anterior and posterior capsules once lens 10 is implanted in an eye. In addition, the “T” shape of haptics 14 is places joint 22 in the same radial location as contact point 25 of haptics 14 with the capsule bag, thereby allowing the anterior capsule remnant to fall posteriorly and contact the posterior capsule, possibly reducing the incidence of posterior capsule opacification. As best seen in FIG. 3, haptics 14 are thinner (in the plane parallel to the plane of optic 12) in the area around joints 22 than distal portion 23, and portions 23 are thicker in the plane of optic 12 than in the plane perpendicular to the plane of optic 12, this assures that haptics 14 will flex at joints 22, causing lens 10 to vault. In addition, distal portions 23 of haptics 14 are generally scalloped-shaped. This assures that distal portions 23 of haptics 14 will flex inwardly and conform to the shape of the capsular bag. While any suitable dimensions can be used, haptics 14 preferably are between about 0.10 mm and 0.40 mm thick, with around 0.20 mm being most preferred. While lens 10 is illustrated having three haptics 14, once skilled in the art will recognize that lens 10 may contain two or more haptics 14, provided that there are sufficient haptics 14 to contact the majority of the equator of the capsular bag along at least 220°. Such a broad area of contact reduces the stress induced by the lens to the capsular bag while keeping the capsular bag in equal tension, thereby minimizing the formation of folds in the capsular bag and helping the capsular bag to keep a round shape. The soft construction of lens 10, and in particular, joints 22, allows lens 10 to be compressed by the capsular bag (e.g., to around 10 millimeters), thereby tightening the zonules and facilitating accommodation.

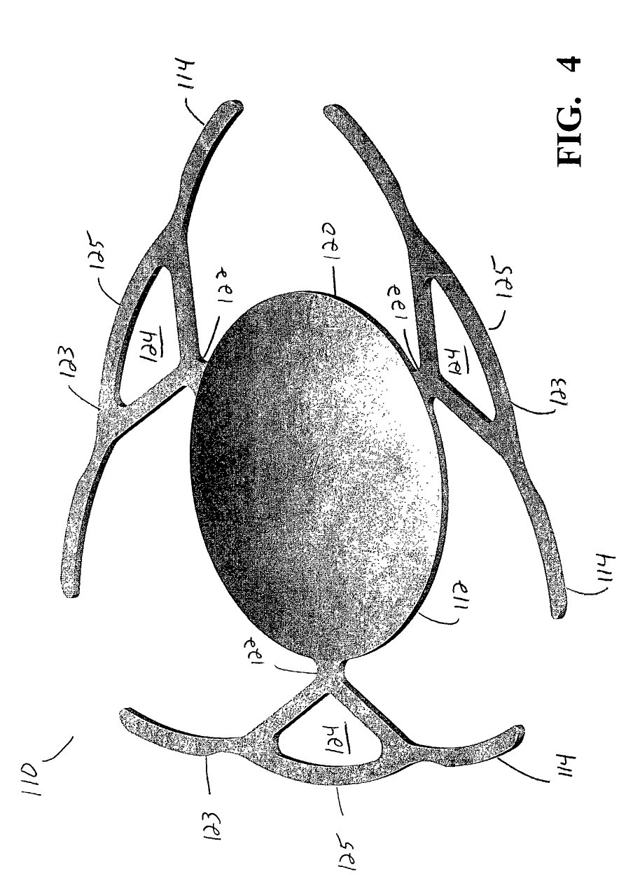

As best seen in FIG. 4, in a second embodiment of the present invention, lens 110 generally consists of optic 112 and a plurality of haptics 114. Lens 110 may have any suitably designed optic 112 (e.g., multifocal, toric, monofocal) and preferably made in a single piece from a soft, foldable material, such as silicone, hydrogel or soft acrylic and symmetrical about the optical axis. Haptics 114 intersects edge 120 of optic 112 at an angle relative to the plane of the optic, preferably between 1° and 20°, and most preferably between 3° and 10° but other suitable angles may also be used. Haptics 114 preferably are generally “T”-shaped in the plane of optic 112, attached to optic 112 at joints 122 and contain open areas 124 that reduce the mass and increase the flexibility of haptics 114. Open areas 124 also provide for increased contact between the anterior and posterior capsules once lens 110 is implanted in an eye. In addition, the “T” shape of haptics 114 places joint 122 in the same radial location as contact point 125 of haptics 114 with the capsule bag, thereby allowing the anterior capsule remnant to fall posteriorly and contact the posterior capsule, possibly reducing the incidence of posterior capsule opacification. Distal portions 123 of haptics 114 are generally scalloped-shaped. This assures that distal portions 123 of haptics 114 will flex inwardly and conform to the shape of the capsular bag. Lens 110 is generally of the same design and construct as lens 10 except that haptics 114 are of relatively constant thickness from joints 122 to contact points 125.

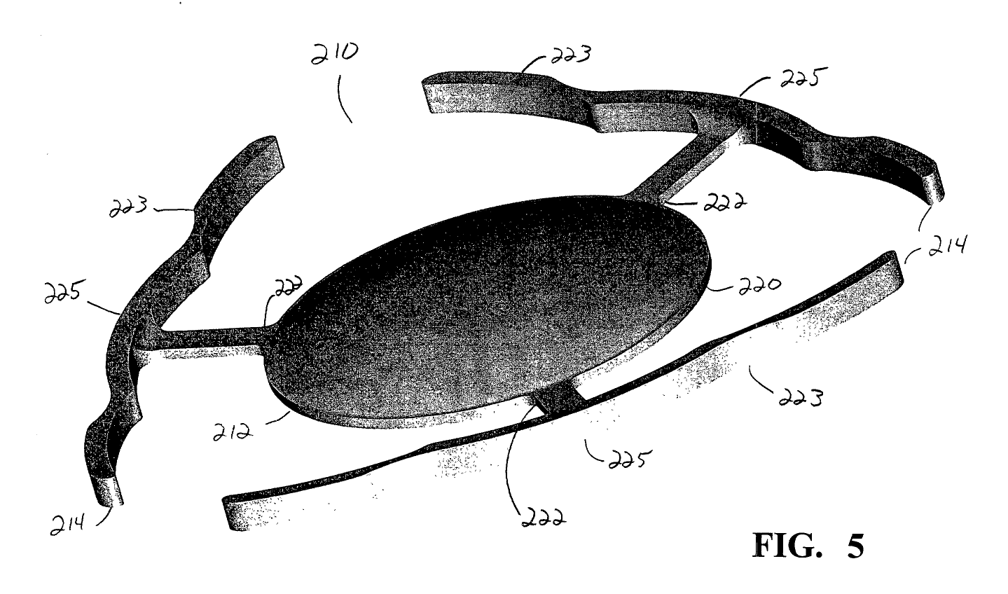

As best seen in FIG. 5, in a third embodiment of the present invention, lens 210 generally consists of optic 212 and a plurality of haptics 214. Lens 210 may have any suitably designed optic 212 (e.g., multifocal, toric, monofocal) and preferably made in a single piece from a soft, foldable material, such as silicone, hydrogel or soft acrylic and symmetrical about the optical axis. Haptics 214 intersects edge 220 of optic 212 at an angle relative to the plane of the optic, preferably between 1° and 20°, and most preferably between 3° and 10° but other suitable angles may also be used. Haptics 214 preferably are generally “T”-shaped in the plane of optic 212, attached to optic 212 at joints 222 and are thinner (in the plane of optic 212) in the area around joints 222 than distal portion 223, and portions 223 are thicker in the plane of optic 212 than in the plane perpendicular to the plane of optic 212. This assures that haptics 214 will flex at joints 222, causing lens 210 to vault, and that distal portions 223 of haptics 214 will flex inwardly and conform to the shape of the capsular bag. In addition, the “T” shape of haptics 214 places joint 222 in the same radial location as contact point 225 of haptics 214 with the capsule bag, thereby allowing the anterior capsule remnant to fall posteriorly and contact the posterior capsule, possibly reducing the incidence of posterior capsule opacification. Distal portions 223 of haptics 214 are generally scalloped-shaped. This assures that distal portions 223 of haptics 214 will flex inwardly and conform to the shape of the capsular bag. Lens 210 is generally of the same design and construct as lens 10 except that haptics 214 do not contain open areas 24.

While certain embodiments of the present invention have been described above, these descriptions are given for purposes of illustration and explanation. Variations, changes, modifications, and departures from the systems and methods disclosed above may be adopted without departure from the scope or spirit of the present invention.

I claim:

1. An intraocular lens, comprising:
a) an optic made from a foldable material; and
b) at least one open-loop haptic integrally formed with the optic as a single piece, the haptic intersecting the optic at an angle relative to the plane of the optic, wherein the haptic is generally “T”-shaped and attaches to the optic at a joint.

2. The intraocular lens of claim 1 wherein the lens comprises three haptics.

3. The intraocular lens of claim 1 wherein the haptic contains a void.

4. The intraocular lens of claim 1 wherein the haptic further contains a distal portion located distally of the joint and the distal portion is thicker in the plane of the optic than the joint.

5. The intraocular lens of claim 4 wherein the distal portions are generally scalloped-shaped.

6. The intraocular lens of claim 1 wherein the haptic further contains a distal portion located distally of the joint and the distal portion is generally scallop-shaped.

7. The intraocular lens of claim 1 wherein the haptic is generally of a constant thickness.

8. An intraocular lens, comprising:
a) an optic made from a foldable material; and
b) at least one open-loop haptic integrally formed with the optic as a single piece, the haptic intersecting the optic at an angle relative to the plane of the optic, wherein the haptic is generally “T”-shaped and attaches to the optic at a joint so that compression of the haptics causes the optic to move along an optical axis.

9. The intraocular lens of claim 8 wherein the lens comprises three haptics.

10. The intraocular lens of claim 8 wherein the haptic contains a void.

11. The intraocular lens of claim 8 wherein the haptic further contains a distal portion located distally of the joint and the distal portion is thicker in the plane of the optic than the joint.

12. The intraocular lens of claim 8 wherein the haptic is generally of a constant thickness.

13. An intraocular lens, comprising:
a) an optic made from a foldable material; and
b) at least one open-loop haptic integrally formed with the optic as a single piece, the haptic intersecting the optic at an angle relative to the plane of the optic, wherein the haptic is generally “T”-shaped and attaches to the optic at a joint located in the same radial location as a contact point of the haptic with a capsule bag.

14. The intraocular lens of claim 13 wherein the lens comprises three haptics.

15. The intraocular lens of claim 13 wherein the haptic contains a void.

16. The intraocular lens of claim 13 wherein the haptic further contains a distal portion located distally of the joint and the distal portion is thicker in the plane of the optic than the joint.

17. The intraocular lens of claim 16 wherein the distal portions are generally scalloped-shaped.

18. The intraocular lens of claim 13 wherein the haptic further contains a distal portion located distally of the joint and the distal portion is generally scallop-shaped.

19. The intraocular lens of claim 13 wherein the haptic is generally of a constant thickness.
